(19) United States
(12) Patent Application Publication
(10) Pub. No.: US 2020/0294682 A1
(43) Pub. Date: Sep. 17, 2020

(54) MEDICAL INTERVIEW APPARATUS

(71) Applicant: CANON MEDICAL SYSTEMS CORPORATION, Otawara-shi (JP)

(72) Inventor: Masaki YOSHIDA, Yaita (JP)

(73) Assignee: CANON MEDICAL SYSTEMS CORPORATION, Otawara-shi (JP)

(21) Appl. No.: 16/817,395

(22) Filed: Mar. 12, 2020

(30) Foreign Application Priority Data
Mar. 13, 2019 (JP) 2019-045374
Mar. 11, 2020 (JP) 2020-042165

(51) Int. Cl. G16H 80/00; G16H 50/20; G16H 10/20

(52) U.S. Cl. CPC G16H 80/00; G16H 10/20; G16H 50/20

(57) ABSTRACT

A medical interview apparatus according to an embodiment includes: processing circuitry configured to receive an initial answer to an initial medical questionnaire inquiring a symptom; present an additional medical questionnaire inquiring a symptom related to the initial answer based on the initial answer; receive an additional answer to the additional medical questionnaire; and determine at least one first disease name based on the initial answer and the additional answer.

### CROSS-REFERENCE TO RELATED APPLICATIONS

This application is based upon and claims the benefit of priority from Japanese Patent Application No. 2019-045374, filed 2019 Mar. 13 and Japanese Patent Application No. 2020-042165, filed 2020 Mar. 11; the entire contents of all of which are incorporated herein by reference.

### FIELD

Embodiments described herein relate generally to a medical interview apparatus.

### BACKGROUND

Before consulting a doctor at a hospital, a patient usually receives a previously prepared medical questionnaire at a hospital reception and enters his/her symptoms in the medical questionnaire.

Such a previously prepared medical questionnaire is created for an unspecified large number of patients, and the patient may have difficulty in correctly entering his/her real symptoms in the previously prepared medical questionnaire. Thus, a system that generates a new question item in response to a medical questionnaire answer and asks a patient again has been proposed.

Unfortunately, such a conventional system simply asks a symptom answered by the patient in more detail as the new question item and does not efficiently inquire the symptoms of the patient.

### BRIEF DESCRIPTION OF THE DRAWINGS

FIG. 1 is a system block diagram illustrating a configuration example of a medical interview system according to an embodiment;

FIG. 2 is a functional block diagram illustrating a functional configuration example of a user terminal and a doctor terminal;

FIG. 3 is a functional block diagram illustrating a functional configuration example of a server apparatus according to the embodiment;

FIG. 4 is a view illustrating an example of an initial medical questionnaire;

FIG. 5 is a view illustrating an example of correspondence relations between symptoms and disease names stored in a disease name symptom database;

FIG. 6 is a view illustrating an example of selecting symptoms desired to be additionally inquired;

FIG. 7 (FIG. 7A and FIG. 7B) is a view illustrating an example of an additional medical questionnaire;

FIG. 8 (FIG. 8A and FIG. 8B) is a view illustrating a display example of symptoms obtained by a medical interview, displayed on the doctor terminal by the server apparatus;

FIG. 9 is a view illustrating an example of medical interview information displayed on the doctor terminal by the server apparatus;

FIG. 10 is a flowchart illustrating an example of a processing flow of the medical interview system; and

FIG. 11 is a view for explaining a medical interview system 5 according to a second embodiment.

### DETAILED DESCRIPTION

According to one embodiment, a medical interview apparatus comprises processing circuitry. The processing circuitry is configured to receive an initial answer to an initial medical questionnaire inquiring a symptom; present an additional medical questionnaire inquiring a symptom related to the initial answer based on the initial answer; receive an additional answer to the additional medical questionnaire; and determine at least one first disease name based on the initial answer and the additional answer.

Embodiments of a medical interview apparatus and a medical interview system will be described below with reference to the drawings.

### First Embodiment

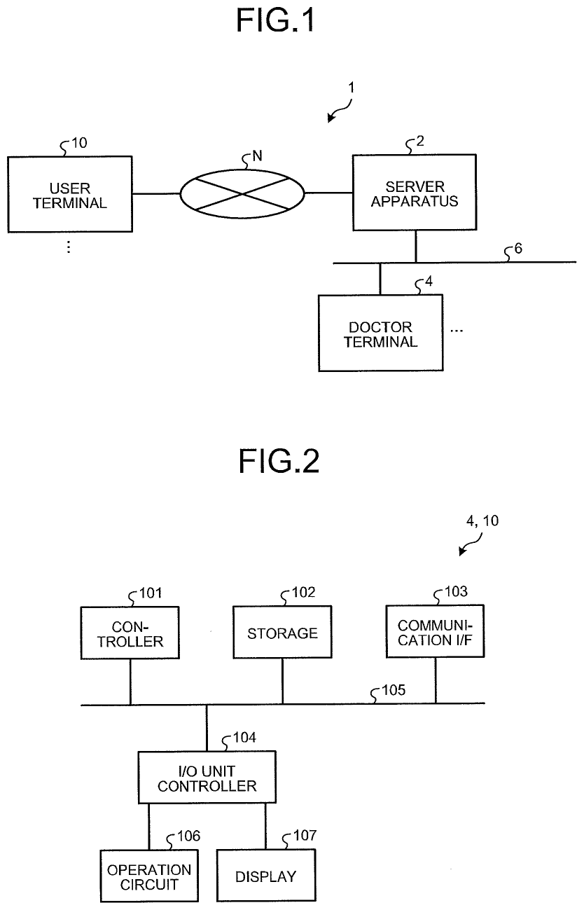

FIG. 1 is a view illustrating a system configuration example of a medical interview system according to an embodiment.

Explanation of an Entire Configuration of the Medical Interview System

As illustrated in FIG. 1, a medical interview system 1 according to the present embodiment includes a server apparatus 2, a doctor terminal 4, and a user terminal 10. The server apparatus 2 and the user terminal 10 are communicably connected to each other via a communication network N. The communication network N is, for example, the Internet or a private network such as a virtual private network (VPN). The server apparatus 2 and the doctor terminal 4 can directly or indirectly communicate with each other, for example, by a hospital local area network (LAN) 6 installed in a hospital. The user terminal 10, which is possessed by an individual patient, may include a plurality of user terminals. The doctor terminal 4, which is installed, for example, in an individual consultation room, may also include a plurality of doctor terminals 4 if there are a plurality of consultation rooms.

The server apparatus 2 receives an initial answer to an initial medical questionnaire inquiring symptoms from the user terminal 10. The server apparatus 2 presents an additional medical questionnaire inquiring a symptom related to the initial answer to the user terminal 10 based on the initial answer. The server apparatus 2 receives an additional answer to the additional medical questionnaire from the user terminal 10, and determines at least one first disease name based on the initial answer and the additional answer. The term “determine” in the present embodiment includes prediction using an AI model and identification by an arithmetic operation using a table.

The server apparatus 2 also transmits the initial medical questionnaire inquiring symptoms to the user terminal 10. The server apparatus 2 receives an answer to the initial medical questionnaire from the user terminal 10, and predicts at least one disease name based on a symptom answered in the initial medical questionnaire. The server apparatus 2 predicts another symptom from the predicted disease name, and generates the additional medical questionnaire inquiring a symptom to be asked again in accordance with the predicted other symptom. The server apparatus 2 may acquire an additional medical questionnaire in accordance with the answer to the initial medical questionnaire from additional medical questionnaires that have already been created. The server apparatus 2 then transmits the additional medical questionnaire to the user terminal 10, and receives an answer to the additional medical questionnaire from the user terminal 10. The server apparatus 2 further narrows down a disease name candidate based on the answer to the additional medical questionnaire. The server apparatus 2 is an example of the medical interview apparatus. The user terminal 10 is an example of a terminal apparatus.

The doctor terminal 4 is a terminal such as a personal computer installed, for example, in an individual consultation room. The doctor terminal 4 receives medical interview information (medical interview results) including the answers to the initial medical questionnaire and the additional medical questionnaire, and the predicted disease name predicted by the server apparatus 2 from the server apparatus 2, and displays the received medical interview information. A doctor examines a target patient by referring to the medical interview information, and inputs medical record information of an electronic medical record, that is, the symptoms of the patient and the doctor's opinion from the doctor terminal 4.

The user terminal 10 receives the initial medical questionnaire and the additional medical questionnaire from the server apparatus 2. The user terminal 10 receives the answers to the initial medical questionnaire and the additional medical questionnaire from, for example, the patient. The user terminal 10 also transmits the answers to the initial medical questionnaire and the additional medical questionnaire to the server apparatus 2. The user terminal 10 is, for example, a portable terminal such as a smartphone and a tablet personal computer (PC) possessed by the patient or his/her family.

Explanation of a Functional Configuration of the User Terminal and the Doctor Terminal

FIG. 2 is a functional block diagram illustrating a functional configuration example of the user terminal 10 and the doctor terminal 4. As illustrated in FIG. 2, the user terminal 10 and the doctor terminal 4 are configured such that a controller 101, a storage 102, a communication interface (I/F) 103, and an input/output (I/O) unit controller 104 are connected via a bus 105. The user terminal 10 may be any device that at least has functions of displaying the medical questionnaire, filling out the medical questionnaire, and transmitting and receiving the medical questionnaire via a network or a communication line. The user terminal 10 may be, for example, a smartphone. The doctor terminal 4 may be any device that at least has functions of browsing the medical questionnaire and transmitting and receiving the medical questionnaire via a network or a communication line. The doctor terminal 4 may be, for example, a medical record terminal.

The controller 101 has functions as a processor and is composed of a central processing unit (CPU), a read-only memory (ROM), a random-access memory (RAM), or the like. The controller 101 controls each unit of the doctor terminal 4 or the user terminal 10 with the CPU reading a control computer program stored in the ROM or the storage 102, loading the computer program into the RAM, and sequentially executing the computer program. The storage 102 is, for example, a hard disk drive, and stores the control computer program executed by the CPU of the controller 101 and various types of setting data. Examples of the control computer program stored in the storage 102 include a browser for intercommunication with the server apparatus 2, and a file communication application for transmitting and receiving a medical questionnaire file to and from the server apparatus 2.

The communication I/F 103 controls communication between the user terminal 10 and the server apparatus 2, and between the doctor terminal 4 and the server apparatus 2. To be more specific, the communication I/F 103 of the user terminal 10 performs data communication with the server apparatus 2 via the communication network N. The communication I/F 103 of the doctor terminal 4 performs data communication with the server apparatus 2 via a hospital LAN 6.

The I/O unit controller 104 controls various I/O units under the control of the controller 101. Examples of the I/O units controlled by the I/O unit controller 104 include an operation circuit 106, such as a keyboard, a mouse and a touch panel, and a display 107, such as a liquid crystal display.

In particular, in the user terminal 10, the communication I/F 103 receives the initial medical questionnaire and the additional medical questionnaire from the server apparatus 2. The communication I/F 103 transmits the answer (the initial answer) for the initial medical questionnaire and the answer (the additional answer) for the additional medical questionnaire to the server apparatus 2. The communication I/F 103 is an example of a receiver and a transmitter.

In the user terminal 10, the display 107 displays the initial medical questionnaire and the additional medical questionnaire. The controller 101 receives the answers to the initial medical questionnaire and the additional medical questionnaire from the patient. The controller 101 is an example of a receiver.

Explanation of a Functional Configuration of the Server Apparatus

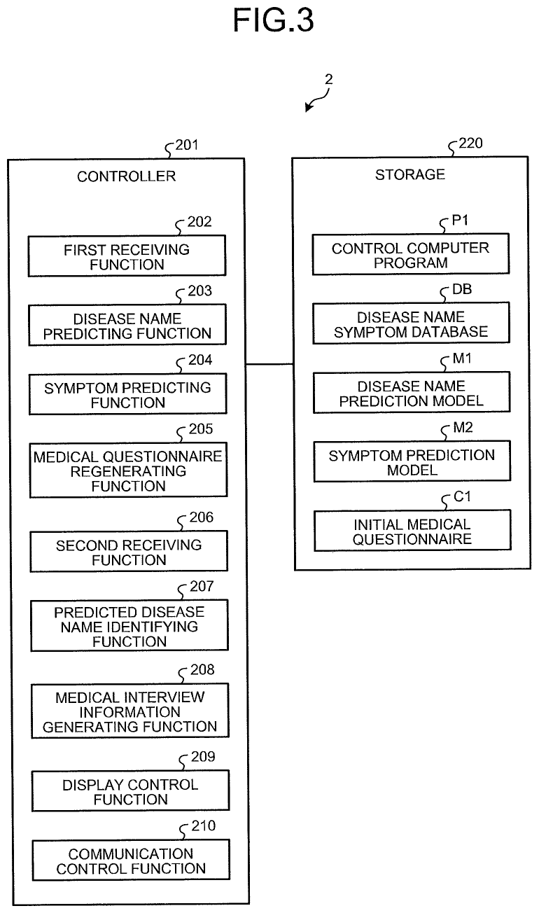

FIG. 3 is a functional block diagram illustrating a functional configuration example of the server apparatus 2. As illustrated in FIG. 3, the server apparatus 2 includes a controller 201 and a storage 220.

The controller 201 has functions as a processor and is composed of a CPU, a ROM, a RAM, or the like. The controller 201 controls each unit of the server apparatus 2 with the CPU loading a control computer program P1 stored in the ROM or the storage 220 into the RAM and sequentially executing the computer program.

By the CPU sequentially executing the control computer program P1, the controller 201 achieves, as functional parts, a first receiving function 202, a disease name predicting function 203, a symptom predicting function 204, a medical questionnaire regenerating function 205, a second receiving function 206, a predicted disease name identifying function 207, a medical interview information generating function 208, a display control function 209 and a communication control function 210 as illustrated in FIG. 3.

The first receiving function 202 receives an answer to an initial medical questionnaire C1 inquiring symptoms. To be more specific, the server apparatus 2 receives the completed initial medical questionnaire C1 from the user terminal 10. The first receiving function 202 may receive the initial medical questionnaire C1 every time a user filling out the initial medical questionnaire C1 operates, for example, a “Next” button to move to a next question item of the initial medical questionnaire C1 in the user terminal 10. When detecting activation of an application for filling out the initial medical questionnaire C1 in the user terminal 10, or when detecting input of a hospital name in the user terminal 10, the server apparatus 2 determines that the medical questionnaire has been requested, and makes the communication control function 210 transmit the initial medical questionnaire C1 to the user terminal 10.

The disease name predicting function 203 predicts (or determines) at least one predicted disease name based on the answer received by the first receiving function 202. The disease name predicting function 203 predicts at least one predicted disease name as a first or second disease name based on a symptom of a patient (the initial answer) entered in the initial medical questionnaire C1 received by the first receiving function 202. To be more specific, the disease name predicting function 203 predicts at least one predicted disease name by using a table in which an individual symptom and a disease name causing the symptom correspond to each other. The disease name predicting function 203 also predicts at least one predicted disease name by using a pre-trained model outputting at least one second disease name for at least one symptom input thereto. The disease name predicting function 203 is an example of a first or second determining part.

The symptom predicting function 204 predicts a symptom of the predicted disease name predicted by the disease name predicting function 203 based on the predicted disease name. When the disease name predicting function 203 predicts only one predicted disease name, the symptom predicting function 204 predicts another symptom possibly corresponding to the predicted disease name.

The medical questionnaire regenerating function 205 generates an additional medical questionnaire C2 inquiring a symptom in accordance with the symptom predicted by the symptom predicting function 204. The medical questionnaire regenerating function 205 also highlights the symptom predicted by the symptom predicting function 204 in the generated additional medical questionnaire C2. When the disease name predicting function 203 predicts only one predicted disease name, the medical questionnaire regenerating function 205 generates the additional medical questionnaire C2 inquiring another symptom possibly corresponding to the predicted disease name. The medical questionnaire regenerating function 205 and the symptom predicting function 204 are an example of a first generating part. That is, the medical questionnaire regenerating function 205 and the symptom predicting function 204 generate the additional medical questionnaire by using a pre-trained model outputting a medical questionnaire for at least one disease name input thereto. The medical questionnaire regenerating function 205 and the symptom predicting function 204 also generate the additional medical questionnaire by an arithmetic operation using a table in which a plurality of disease names and a plurality of medical interviews correspond to each other.

When the disease name predicting function 203 predicts a plurality of predicted disease names, the medical questionnaire regenerating function 205 generates the additional medical questionnaire C2 including a question regarding a symptom predicted to have a highest onset possibility out of symptoms predicted from the respective predicted disease names by the symptom predicting function 204.

The medical questionnaire regenerating function 205 may classify the onset possibility, for example, into five levels from 1, which is lowest, to 5, which is highest, and add a question regarding a symptom having an onset possibility higher than a predetermined level to the additional medical questionnaire C2.

When the disease name predicting function 203 predicts a plurality of predicted disease names, the medical questionnaire regenerating function 205 generates the additional medical questionnaire C2 including a question regarding a symptom more shared among the predicted disease names out of symptoms predicted from the respective predicted disease names by the symptom predicting function 204.

When the disease name predicting function 203 predicts a plurality of predicted disease names, the medical questionnaire regenerating function 205 generates the additional medical questionnaire C2 including a question regarding a symptom less shared among the predicted disease names out of symptoms predicted from the respective predicted disease names by the symptom predicting function 204.

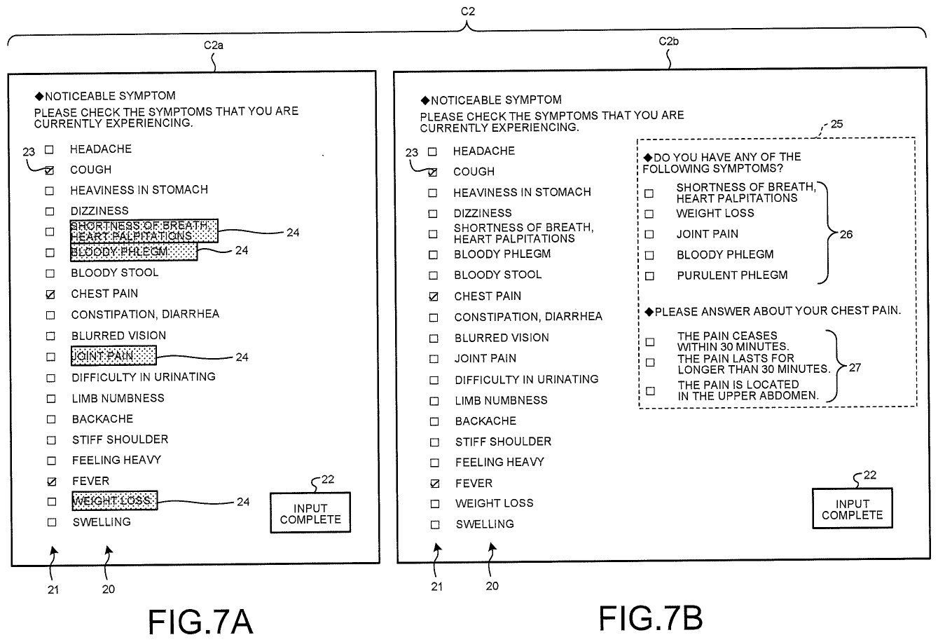

The medical questionnaire regenerating function 205 may acquire the additional medical questionnaire C2 in accordance with the answer to the initial medical questionnaire C1 from the additional medical questionnaires C2 that have already been created. A specific example of the additional medical questionnaire C2 will be described later (FIG. 7A and FIG. 7B).

The second receiving function 206 receives an answer to the additional medical questionnaire C2 generated by the medical questionnaire regenerating function 205.

The predicted disease name identifying function 207 identifies (or determines) the predicted disease name predicted by the disease name predicting function 203 as the first or second disease name based on the initial answer to the initial medical questionnaire and the symptom received as the additional answer by the second receiving function 206. That is, the predicted disease name identifying function 207 identifies (or determines) the predicted disease name predicted by the disease name predicting function 203 as the first or second disease name by using a table in which an individual symptom and a disease name causing the symptom correspond to each other. The predicted disease name identifying function 207 also identifies (or determines) the predicted disease name predicted by the disease name predicting function 203 as the first or second disease name by using a pre-trained model outputting at least one disease name for at least one symptom input thereto. Additionally, when the disease name predicting function 203 predicts only one predicted disease name, the predicted disease name identifying function 207 determines whether the predicted disease name is developed based on the answer to the additional medical questionnaire C2.

The predicted disease name identifying function 207 is an example of the first or second determining part.

The medical interview information generating function 208 generates medical interview information 35 including the answer (the initial answer) for the initial medical questionnaire C1, the answer (the additional answer) for the additional medical questionnaire C2, and the disease name identified by the predicted disease name identifying function 207. The medical interview information generating function 208 is an example of a second generating part. In the medical interview information 35, the symptom characterizing the disease name identified by the predicted disease name identifying function 207 is highlighted.

The display control function 209 displays the initial medical questionnaire C1 and the additional medical questionnaire C2 on the display 107 of the user terminal 10. The display control function 209 also displays the medical interview information 35 on the display 107 of the doctor terminal 4 to which a doctor inputs medical record information.

The communication control function 210 controls communication between the controller 201 and the storage 220. The communication control function 210 also controls communication between the server apparatus 2 and the user terminal 10, and between the server apparatus 2 and the doctor terminal 4.

The storage 220 is, for example, a hard disk drive, and stores the control computer program P1 executed by the CPU of the controller 201 and various types of data. The storage 220 also stores a disease name symptom database DB, a disease name prediction model M1, a symptom prediction model M2, and the initial medical questionnaire C1.

The control computer program P1 executed by the controller 201 may be recorded as an installable or executable file in a computer-readable recording medium such as a compact disc read-only memory (CD-ROM), a flexible disk (FD), a compact disc-recordable (CD-R), and a digital versatile disc (DVD).

The control computer program P1 may be stored in a computer connected to a network such as the Internet such that the control computer program P1 is downloaded via the network. Alternatively, the control computer program P1 may be delivered or distributed via a network such as the Internet.

The disease name symptom database DB is a database that manages disease names and symptoms of various diseases. The disease name symptom database DB is created based on medical examination information of doctors at an individual hospital or guidelines issued by a learned society or the like.

The disease names and the symptoms stored in the disease name symptom database DB are associated with each other. For example, an ID uniquely assigned to each disease name is associated with a symptom that is a chief complaint of the disease name in the disease name symptom database DB. An ID is also uniquely assigned to each symptom and is associated with a disease name causing the symptom.

The disease name prediction model M1 is a model that outputs one or more disease names for at least one symptom. The disease name prediction model M1 can be achieved by a first model (AI model) in which an individual symptom, a disease name causing the symptom, and information indicating certainty of association between the symptom and the disease name are stored corresponding to one another, and a second model that executes an arithmetic operation using a table in which an individual symptom and a disease name causing the symptom are stored corresponding to each other.

The disease name prediction model M1 as the first model is a computer program achieving an AI model such as a neural network. That is, the disease name prediction model M1 is, for example, a pre-trained model (such as a deep neural network) outputting one or more disease names for at least one symptom input thereto. The computer program achieving the disease name prediction model M1 can be constructed by machine learning using a plurality of sets of training data having a combination of at least one symptom as a typical chief complaint of a patient having a certain disease name, and the disease name diagnosed by a doctor.

The disease name prediction model M1 as the second model is an arithmetic computer program determining one or more disease names based on at least one symptom by using a previously created table defining a plurality of correspondence relations between disease names and symptoms.

Both of the first model and the second model of the disease name prediction model M1 can be formed using, for example, the data stored in the disease name symptom database DB. The information indicating certainty of association between the symptom and the disease name may be calculated, for example, based on the number of samples used for the learning of the disease name prediction model M1. To be more specific, when M patients out of N patients having a symptom of “cough” are diagnosed as “pneumonia”, certainty of predicting pneumonia from cough is registered as M/N. The certainty is changed with update of the disease name prediction model M1.

The symptom prediction model M2 is a model (information) that stores association of an individual disease name, a symptom corresponding to the disease name, and information indicating certainty of association between the disease name and the symptom.

The symptom prediction model M2 is a model that outputs one or more symptoms for at least one disease name. The symptom prediction model M2 can be achieved by a third model (AI model) in which an individual disease name, at least one symptom corresponding to a disease of the disease name, and information indicating certainty of association between the disease name and the symptom are stored corresponding to one another, and a fourth model that executes an arithmetic operation using a table in which an individual disease name and a symptom corresponding to a disease of the disease name are stored corresponding to each other.

The symptom prediction model M2 as the third model is a computer program achieving an AI model such as a neural network. That is, the symptom prediction model M2 may be, for example, a pre-trained model (such as a deep neural network) outputting one or more symptoms for at least one disease name input thereto. The computer program achieving the symptom prediction model M2 can be constructed by machine learning using a plurality of sets of training data having a combination of a disease name diagnosed by a doctor and at least one symptom as a typical chief complaint of a patient having the disease name.

The symptom prediction model M2 as the fourth model is an arithmetic computer program determining one or more symptoms based on at least one disease name by using a previously created table defining a plurality of correspondence relations between symptoms and disease names.

Both of the third model and the fourth model of the symptom prediction model M2 can be formed using, for example, the data stored in the disease name symptom database DB. The information indicating certainty of association between the disease name and the symptom may be calculated, for example, based on the number of samples used for the learning of the symptom prediction model M2. To be more specific, when M patients out of N patients are diagnosed as “pneumonia” have a symptom of “cough”, certainty of predicting cough from pneumonia is registered as M/N. The certainty is changed with update of the symptom prediction model M2.

The disease name prediction model M1 and the symptom prediction model M2 may be created and updated by using existing clinical data, or by using data collected by operating the medical interview system 1 of the present embodiment, that is, correspondence relations between entry contents of medical questionnaires and actual diagnostic results.

The disease name prediction model M1 and the symptom prediction model M2 have a learning function. That is, when the disease name predicted by the server apparatus 2 is different from a disease name diagnosed by a doctor, the server apparatus 2 updates the disease name prediction model M1 and the symptom prediction model M2 by associating the symptom of the patient with the disease name diagnosed by the doctor by a model updating part not illustrated in FIG. 3.

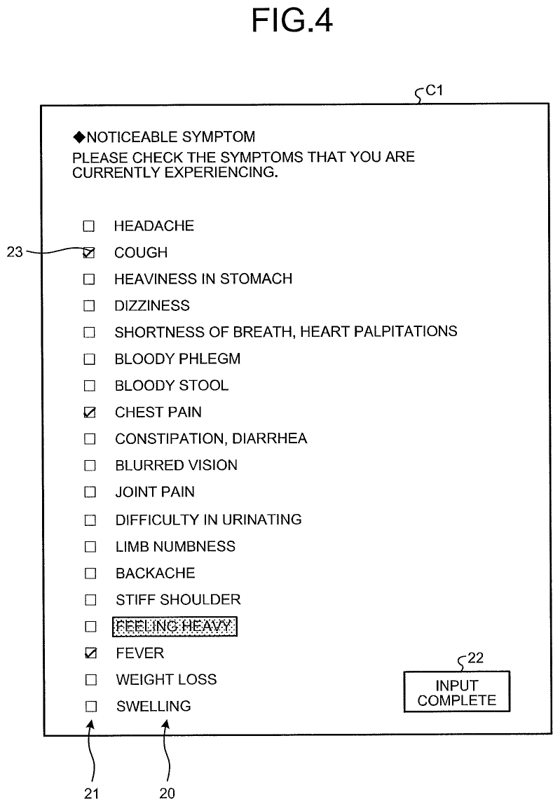

The initial medical questionnaire C1 is a medical questionnaire initially presented to the patient using the user terminal 10 and inquiring the symptoms of the patient. The initial medical questionnaire C1 is described below in detail (FIG. 4).

Explanation of the Initial Medical Questionnaire

FIG. 4 is a view illustrating an example of the initial medical questionnaire C1. The initial medical questionnaire C1 includes symptom information 20, check boxes 21, and an input complete button 22. The patient checks the initial medical questionnaire C1 displayed on the display 107 of the user terminal 10. The patient operates the operation circuit 106 (for example, a touch panel) to input a check mark 23 to a check box 21 corresponding to symptom information 20 related to his/her symptom out of the symptom information 20 displayed in the initial medical questionnaire C1. To be more specific, the controller 101 of the user terminal 10 displays the check mark 23 in the check box 21 touched by the patient.

After inputting all of his/her symptoms, the patient pushes the input complete button 22. The controller 101 of the user terminal 10 detects that the input complete button 22 has been pushed, and transmits the initial medical questionnaire C1 to the server apparatus 2.

The initial medical questionnaire C1 is previously created in order to comprehensively check the symptoms of patients, and is stored in the storage 220 of the server apparatus 2. The initial medical questionnaire C1 has question items regarding various physical symptoms so as to comprehensively check the symptoms of patients.

The initial medical questionnaire C1 dedicated for a specific department (e.g., otolaryngology, ophthalmology, and orthopedics) may be prepared in the storage 220 for a patient who wants to consult a doctor in a specific department.

Although the initial medical questionnaire C1 in FIG. 4 is answered by inputting the check mark 23 to the check box 21, the answer format is not limited to this method. That is, the patient may input his/her symptom by a word (for example, headache), or by a sentence (for example, I have a headache).

The patient may further input the degree of his/her symptom by a numerical value (for example, ten levels). The first receiving function 202 of the server apparatus 2 receiving the entry result of the initial medical questionnaire C1 is configured to be able to receive the information input in accordance with the input format of the initial medical questionnaire C1. To be more specific, the server apparatus 2 has, for example, a text recognition function enabling reception of the answer using a word or a sentence.

Explanation of a Method of Predicting a Disease Name Candidate

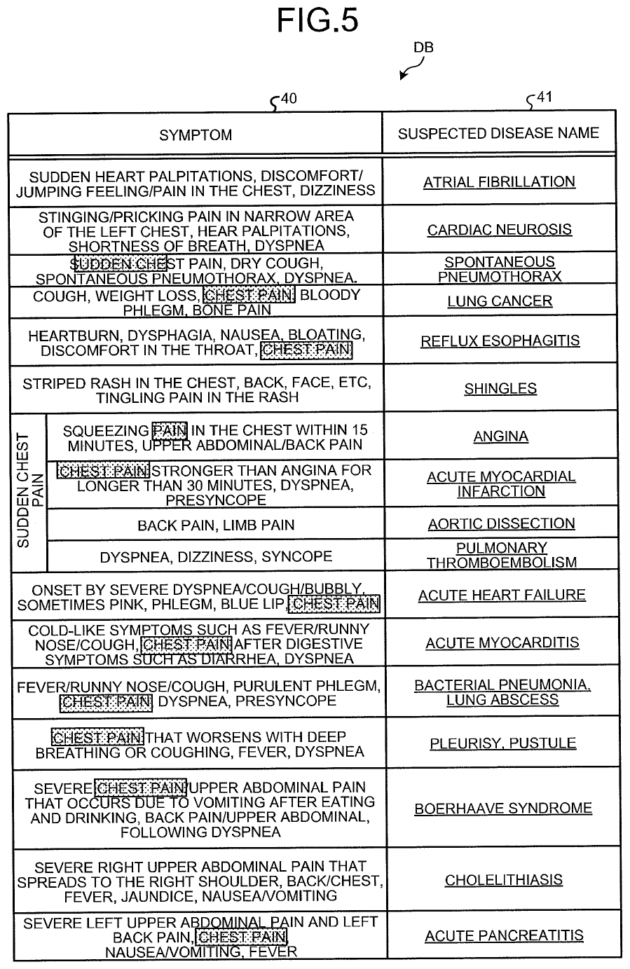

FIG. 5 is a view illustrating an example of correspondence relations between symptoms and disease names stored in the disease name symptom database DB. The disease name symptom database DB stores symptoms 40 and suspected disease names 41 associated with each other.

The disease name predicting function 203 predicts the predicted disease name(s) by using the disease name prediction model M1. For example, the chief complaint of the patient in the initial medical questionnaire C1 is assumed to be “chest pain” as illustrated in FIG. 4. In this case, the disease name predicting function 203 predicts a disease name(s) related to the symptom of “chest pain” as a possible disease name(s) from the column of the symptoms 40 by using the disease name prediction model M1 created based on the disease name symptom database DB.

To be more specific, when the disease name prediction model M1 is described in a table format, the disease name predicting function 203 reads the predicted disease name(s) related to the symptom of “chest pain” from the table. When the disease name prediction model M1 is described as a mathematical model such as a neural network, the disease name predicting function 203 may predict the predicted disease name(s) based on an evaluation value corresponding to the onset possibility of the symptom of “chest pain”, output to an output layer of the neural network for the symptom of “chest pain” input thereto. At this point, the disease name predicting function 203 may output only one predicted disease name outputting a highest evaluation value as a most possible predicted disease name, or may output a plurality of predicted disease names outputting an evaluation value higher than a predetermined threshold value as possible predicted disease names. When the disease name prediction model M1 has the certainty of association between the symptom and the predicted disease name, the predicted disease name(s) to be output may be determined by using the certainty.

In accordance with the example in FIG. 5, spontaneous pneumothorax, lung cancer, reflux esophagitis, angina, acute myocardial infarction, acute heart failure, acute myocarditis, bacterial pneumonia, pleurisy, Boerhaave syndrome, and acute pancreatitis are suspected as the predicted disease names for the symptom of “chest pain” hatched in the drawing.

In the present embodiment, spontaneous pneumothorax, lung cancer, angina, acute myocardial infarction, and bacterial pneumonia are assumed to be predicted as the possible predicted disease names out of the above 11 disease names.

Explanation of a Method of Predicting a Symptom Candidate

The symptom predicting function 204 predicts the symptom(s) by using the symptom prediction model M2. To be more specific, the symptom predicting function 204 predicts whether there is another symptom possibly corresponding to the predicted disease name(s) predicted by the disease name predicting function 203 for the answer in the initial medical questionnaire C1.

The symptom predicting function 204 may predict only one symptom having a highest onset possibility, or a plurality of symptoms having a high onset possibility. At this point, the symptom predicting function 204 possibly predicts the symptom chiefly complained of in the initial medical questionnaire C1, and a symptom not chiefly complained of in the initial medical questionnaire C1. The medical interview system 1 determines to ask the patient again whether the patient has the symptom not chiefly complained of in the initial medical questionnaire C1 out of the symptoms predicted by the symptom predicting function 204.

The symptom not chiefly complained of in the initial medical questionnaire C1 includes a symptom not included in the questions in the initial medical questionnaire C1 and a symptom included in the questions in the initial medical questionnaire C1 but not answered.

The medical questionnaire regenerating function 205 generates the additional medical questionnaire C2 including the symptom(s) desired to be asked again based on the symptom(s) predicted by the symptom predicting function 204. A specific example of the additional medical questionnaire C2 will be described later (FIG. 7A and FIG. 7B).

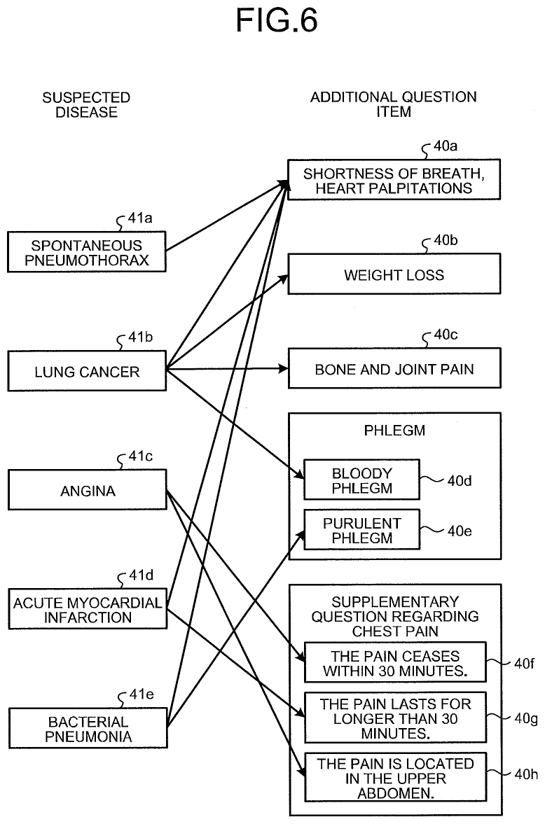

FIG. 6 is a view illustrating an example of selecting symptoms desired to be additionally inquired. FIG. 6 specifically illustrates an example in which additional question items are selected based on the predicted disease names predicted when the patient complains of cough, fever, and chest pain in the initial medical questionnaire C1.

The symptom predicting function 204 predicts symptoms corresponding to the five predicted disease names predicted by the disease name predicting function 203. To be more specific, the symptom predicting function 204 predicts a symptom not included in the questions in the initial medical questionnaire C1 and a symptom included in the questions in the initial medical questionnaire C1 but not answered out of the symptoms predicted from the predicted disease names predicted by the disease name predicting function 203.

FIG. 6 illustrates the example in which the disease name predicting function 203 predicts spontaneous pneumothorax 41a, lung cancer 41b, angina 41c, acute myocardial infarction 41d, and bacterial pneumonia 41e as the predicted disease names as described using FIG. 5.

The symptom predicting function 204 predicts symptoms having a high onset possibility for the respective disease names by using the symptom prediction model M2. The symptom predicting function 204 selects one or a plurality of symptoms having a high onset possibility out of the predicted symptoms.

To be more specific, the symptom predicting function 204 may select a symptom having a highest onset possibility for each of the disease names. The symptom predicting function 204 may output only one symptom predicted to have a highest onset possibility, for example, by using the information indicating certainty of association between the disease name and the symptom stored in the symptom prediction model M2.

When the symptom prediction model M2 is a neural network, the symptom predicting function 204 may predict the symptom(s) based on an evaluation value output to an output layer of the neural network for a specific disease name input thereto. At this point, the symptom predicting function 204 may output only one symptom outputting a highest evaluation value as the symptom having a highest onset possibility.

The symptom predicting function 204 may also output a plurality of symptoms having certainty higher than a predetermined threshold value as the symptoms having a high onset possibility by using the information indicating certainty of association between the disease name and the symptom stored in the symptom prediction model M2. The symptom predicting function 204 may output a plurality of symptoms outputting an evaluation value higher than a predetermined threshold value as the symptoms having a high onset possibility based on the evaluation value output to the output layer of the neural network.

Moreover, the symptom predicting function 204 may output a symptom shared among the disease name candidates as the symptom having a high onset possibility.

The symptom predicting function 204 may output a symptom input many times in the past to the initial medical questionnaire C1 by the same patient as the symptom having a high onset possibility.

Additionally, the symptom predicting function 204 may output a symptom that enables the disease name to be more easily identified out of the symptoms predicted from the predicted disease names.

For example, the symptom predicting function 204 may output a symptom not shared among the disease names. The symptom predicting function 204 may also output a symptom input not many times in the past to the initial medical questionnaire C1 by the same patient.

The symptom predicting function 204 may output a symptom suitable for distinguishing a correct disease name from a wrong disease name out of symptoms expected from both of the correct and wrong disease names if there was a wrong diagnosis in the past. The symptom predicting function 204 may also output a symptom predicted by referring to a past medical history of the patient to add the past medical history to each prediction described above.

The additional question items illustrated in FIG. 6 are symptoms extracted as a result of each prediction described above. That is, shortness of breath, heart palpitations 40a are an example of the symptom more shared among the disease names. Each of weight loss 40b, bone and joint pain 40c, and bloody phlegm 40d is an example of the symptom corresponding only to the lung cancer 41b, that is, the symptom less shared among the disease names.

For phlegm, it is desirable to inquire the condition of phlegm, for example, whether the patient has the bloody phlegm 40d, and purulent phlegm 40e in order to identify the disease name.

Supplementary questions regarding the chest pain, that is, the pain ceases within 30 minutes 40f, the pain lasts for longer than 30 minutes 40g, and the pain is located in the upper abdomen 40h are examples of questions for inquiring the chest pain condition in more detail in order to identify the disease name. Such supplementary questions are also applied to a case, for example, in which a patient complaining of headache is asked about the headache condition (whether the entire headaches, whether a specific area of the headaches, what kind of pain the patient has, how long the patient has had the headache, or the like).

The medical questionnaire regenerating function 205 generates the additional medical questionnaire C2 described below (FIG. 7A and FIG. 7B) based on the additional question items illustrated in FIG. 6.

Explanation of the Additional Medical Questionnaire

FIG. 7 (FIG. 7A and FIG. 7B) is a view illustrating an example of the additional medical questionnaire C2. To be more specific, the left side of FIG. 7 (FIG. 7A) is a view illustrating an example of an additional medical questionnaire C2a, and the right side of FIG. 7 (FIG. 7B) is a view illustrating an example of an additional medical questionnaire C2b.

The additional medical questionnaire C2 follows the display format of the initial medical questionnaire C1 for the sake of patient understandability. It is desirable to highlight the symptom(s) to be asked again, predicted by the symptom predicting function 204. The symptom(s) may be highlighted in any manner, and may be hatched, underlined, displayed in a different color, displayed in a different font, or bolded.

The left side of FIG. 7 (FIG. 7A) is an example of the additional medical questionnaire C2a in which symptoms to be highlighted 24 are hatched.

As another style of highlighting, the symptoms to be asked again may be displayed above the symptom information 20 by rearranging the symptoms. An expected symptom, for example, “Do you have a headache?” may be additionally displayed at a specific position of a screen.

The medical questionnaire regenerating function 205 may also generate the additional medical questionnaire C2b obtained by adding questions to the initial medical questionnaire C1. The right side of FIG. 7 (FIG. 7B) is a view illustrating an example of the additional medical questionnaire C2b. The additional medical questionnaire C2b includes an additional question area 25 added to the initial medical questionnaire C1.

Additional questions 26 and detailed questions 27 are displayed in the additional question area 25. The additional questions 26 are questions determined to be asked again based on the prediction result of the symptom predicting function 204. In the example of the right side of FIG. 7 (FIG. 7B), the additional questions 26 include items included in the questions in the initial medical questionnaire C1 but not answered, and an item not included in the initial medical questionnaire C1. Thus, the additional questions 26 may overlap with the items displayed in the initial medical questionnaire C1. The patient may tick a checkbox in the additional question area 25, or tick the checkbox 21 of the item corresponding to the initial medical questionnaire C1. When one of the checkboxes of the additional question area 25 and the item corresponding to the initial medical questionnaire C1 is ticked, the other checkbox may be ticked as well.

The detailed questions 27 are questions, for example, for obtaining more detailed information about a specific symptom. The questions for inquiring the chest pain condition in detail as described using FIG. 6 are their examples.

When the patient completes the additional medical questionnaire C2 (C2a, C2b), the patient pushes the input complete button 22 similarly to the input of the initial medical questionnaire C1. The user terminal 10 thereby determines that the input of the medical questionnaire has been completed.

The input of the medical questionnaire may be forcibly terminated when the number of symptoms input to the additional medical questionnaire C2 (or the initial medical questionnaire C1) reaches a fixed number. The server apparatus 2 may receive the symptom input from the user terminal 10 and predict the disease name every time the symptom is input, and the input of the medical questionnaire may be forcibly terminated when the number of predicted disease name candidates reaches a fixed number. On the contrary, when the predicted disease name candidates are narrowed down to a fixed number or less, the input of the medical questionnaire may be terminated.

Explanation of a Method of Displaying a Symptom on the Doctor Terminal

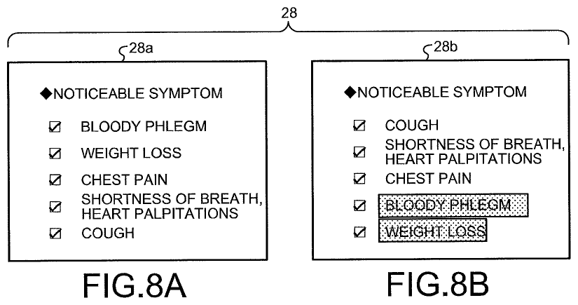

FIG. 8 (FIG. 8A and FIG. 8B) is a view illustrating a display example of symptoms obtained by a medical interview, displayed on the doctor terminal 4 by the server apparatus 2.

The medical interview information generating function 208 arranges the symptoms of the patient in such a form as to allow a medical staff (such as a doctor) who actually examines the patient to easily check the symptoms based on the answers to the initial medical questionnaire C1 and the additional medical questionnaire C2.

The medical interview information generating function 208 generates noticeable symptom information 28 of the patient. The noticeable symptom information 28 is displayed such that the medical staff easily checks the symptoms characterizing the disease name(s) when examining the patient. The left side of FIG. 8 (FIG. 8A) illustrates noticeable symptom information 28a as an example of the noticeable symptom information 28. The noticeable symptom information 28a is an example of highlighting the symptoms characterizing the disease name(s) by displaying the symptoms in descending order of the onset possibility.

To be more specific, the left side of FIG. 8 (FIG. 8A) illustrates the noticeable symptom information 28a having a high possibility of characterizing “lung cancer” that is re-predicted (or determined) by the predicted disease name identifying function 207 based on the answer to the additional medical questionnaire C2. That is, the left side of FIG. 8 (FIG. 8A) is an example in which the symptoms are rearranged and displayed in descending order of the onset possibility during “lung cancer” based on the answers to the initial medical questionnaire C1 and the additional medical questionnaire C2. When the patient develops “lung cancer”, the symptoms complained of by the patient have a decreasing possibility of characterizing lung cancer in the order of “bloody phlegm”>“weight loss”>“chest pain”>“shortness of breath, heart palpitations”>“cough”.

When the predicted disease name identifying function 207 selects a plurality of disease names upon identifying the disease name based on the answer to the additional medical questionnaire C2, the noticeable symptom information 28a may be displayed by ordering the symptoms from a symptom most shared or least shared among the disease name candidates.

Additionally, the noticeable symptom information 28a may be displayed by rearranging the symptoms in descending order of the number of inputs to the initial medical questionnaire C1 and the additional medical questionnaire C2.

The right side of FIG. 8 (FIG. 8B) illustrates noticeable symptom information 28b as an example of the noticeable symptom information 28. The noticeable symptom information 28b is an example of highlighting the symptoms having a high possibility of characterizing “lung cancer” by hatching when the predicted disease name identifying function 207 identifies the disease name based on the answer to the additional medical questionnaire C2, and determines that the disease name is likely to be “lung cancer”. The highlighting method is not limited to hatching, and the symptoms may be underlined, displayed in a different color, displayed in a different font, or bolded.

Explanation of the Medical Interview Information Displayed on the Doctor Terminal

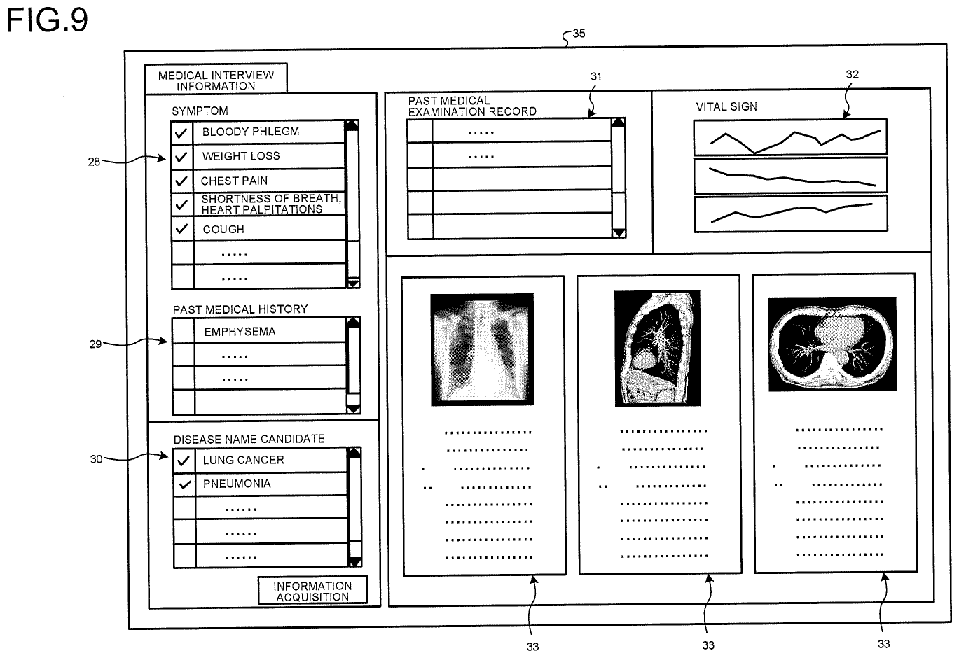

FIG. 9 is a view illustrating an example of the medical interview information 35 displayed on the doctor terminal 4 by the server apparatus 2. The medical interview information 35 includes the noticeable symptom information 28, a past medical history 29, disease name candidates 30, past medical examination records 31, vital signs 32, and diagnostic records 33.

The noticeable symptom information 28 has been described above. In FIG. 9, the symptoms characterizing the predicted disease name candidates 30 are highlighted in descending order of the onset possibility. The past medical history 29 is a history of past illness of the patient. The past medical history can be acquired, for example, by an identification number for identifying an individual patient. The disease name candidates 30 are predicted from the initial medical questionnaire C1 and the additional medical questionnaire C2. FIG. 9 shows that the patient possibly develops lung cancer or pneumonia. For the disease name candidates, only one disease name having a highest possibility may be displayed, or a plurality of disease names having a high possibility may be displayed.

The past medical examination records 31 are information (e.g., a past medical history, past medication information/test information) recorded in electronic medical records during past medical examinations. The vital signs 32 are medical information (e.g., blood test results) related to the disease name(s) (lung cancer and pneumonia in the example of FIG. 9) selected from the disease name candidates 30. The diagnostic records 33 are past diagnostic records of the patient related to the disease name candidates 30. The diagnostic records 33 are acquired from the electronic medical record as information related to the ticked disease names of the disease name candidates 30 and are displayed in the columns of the diagnostic records 33 in FIG. 9 when an information acquisition button provided close to the disease name candidates 30 is pushed. The diagnostic records 33 in FIG. 9 are medical images of the patient captured in the past as one example.

The doctor examines the patient by referring to the medical interview information 35 and diagnoses the disease name of the patient. If the disease names predicted as the disease name candidates 30 in the medical interview information 35 are different from the actual disease name determined by the diagnosis, the medical interview system 1 determines that it made a wrong prediction.

At this point, the server apparatus 2 associates the symptom information input to the initial medical questionnaire C1 and the additional medical questionnaire C2 with the disease name diagnosed by the doctor. The server apparatus 2 then updates the disease name prediction model M1 and the symptom prediction model M2 by using the associated data as new learning data. The learning of the disease name prediction model M1 and the symptom prediction model M2 is desirably performed by giving higher contribution to learning data indicating a relation between the symptom information of the patient obtained by the present diagnosis and the disease name diagnosed by the doctor than other learning data. The learning may be also performed by increasing the contribution of learning data of a disease name group in which the medical interview system 1 made a wrong prediction to the disease name prediction model M1 and the symptom prediction model M2 as compared to that of learning data of other disease name groups.

Explanation of a Processing Flow of the Medical Interview System

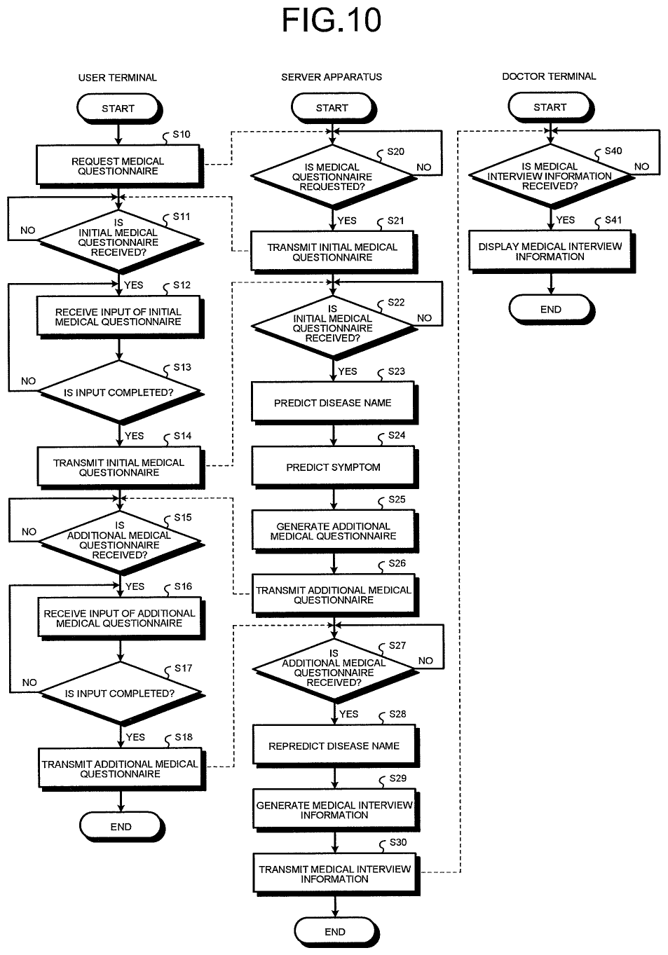

FIG. 10 is a flowchart illustrating an example of a processing flow of the medical interview system 1. A processing flow of the user terminal 10, a processing flow of the server apparatus 2, and a processing flow of the doctor terminal 4 will be sequentially described below.

Explanation of the Processing Flow of the User Terminal

A patient activates an application for executing a medical interview in his/her user terminal 10 and requests a medical questionnaire for the server apparatus 2 (step S10). The patient may input patient information such as a patient name when requesting the medical questionnaire.

The controller 101 of the user terminal 10 determines whether the communication I/F 103 receives the initial medical questionnaire C1 from the server apparatus 2 (step S11). When the controller 101 determines that the initial medical questionnaire C1 has been received (Yes at step S11), the process proceeds to step S12. When the controller 101 does not determine that the initial medical questionnaire C1 has been received (No at step S11), step S11 is repeated.

The controller 101 receives input to the initial medical questionnaire C1 from the patient (step S12).

The controller 101 determines whether the input complete button 22 is pushed by working with the operation circuit 106 (step S13). When the controller 101 determines that the input complete button 22 has been pushed (Yes at step S13), the process proceeds to step S14. When the controller 101 does not determine that the input complete button 22 has been pushed (No at step S13), the process returns to step S12.

When determining that the input complete button 22 has been pushed at step S13, the controller 101 makes the communication I/F 103 transmit the initial medical questionnaire C1 to the server apparatus 2 at the subsequent step S14.

The controller 101 then determines whether the communication I/F 103 receives the additional medical questionnaire C2 from the server apparatus 2 (step S15). When the controller 101 determines that the additional medical questionnaire C2 has been received (Yes at step S15), the process proceeds to step S16. When the controller 101 does not determine that the additional medical questionnaire C2 has been received (No at step S15), step S15 is repeated.

The controller 101 receives input to the additional medical questionnaire C2 from the patient (step S16).

The controller 101 determines whether the input complete button 22 is pushed by working with the operation circuit 106 (step S17). When the controller 101 determines that the input complete button 22 has been pushed (Yes at step S17), the process proceeds to step S18. When the controller 101 does not determine that the input complete button 22 has been pushed (No at step S17), the process returns to step S16.

When determining that the input complete button 22 has been pushed at step S17, the controller 101 makes the communication I/F 103 transmit the additional medical questionnaire C2 to the server apparatus 2 at the subsequent step S18. The user terminal 10 then terminates the process in FIG. 10.

Explanation of the Processing Flow of the Server Apparatus

The first receiving function 202 determines whether the user terminal 10 requests the medical questionnaire (step S20). The server apparatus 2 may acquire, for example, the patient information from the user terminal 10 together with the request for the medical questionnaire at step S20. When the first receiving function 202 determines that the user terminal 10 has requested the medical questionnaire (Yes at step S20), the process proceeds to step S21. When the first receiving function 202 does not determine that the user terminal 10 has requested the medical questionnaire (No at step S20), step S20 is repeated.

When determining that the user terminal 10 has requested the medical questionnaire at step S20, the first receiving function 202 makes the communication control function 210 transmit the initial medical questionnaire C1 to the user terminal 10 at the subsequent step S21.

The first receiving function 202 then determines whether the initial medical questionnaire C1 is received from the user terminal 10 (step S22). When the first receiving function 202 determines that the initial medical questionnaire C1 has been received from the user terminal 10 (Yes at step S22), the process proceeds to step S23. When the first receiving function 202 does not determine that the initial medical questionnaire C1 has been received from the user terminal 10 (No at step S22), step S22 is repeated.

The disease name predicting function 203 predicts the disease name(s) based on the entry contents of the initial medical questionnaire C1 (step S23).

The symptom predicting function 204 predicts the symptom(s) from the disease name(s) predicted at step S23 (step S24).

The medical questionnaire regenerating function 205 generates the additional medical questionnaire C2 based on the symptom(s) predicted at step S24 (step S25).

The medical questionnaire regenerating function 205 makes the communication control function 210 transmit the additional medical questionnaire C2 to the user terminal 10 (step S26).

The predicted disease name identifying function 207 determines whether the additional medical questionnaire C2 is received from the user terminal 10 (step S27). When it is determined that the additional medical questionnaire C2 has been received from the user terminal 10 (Yes at step S27), the process proceeds to step S28. When it is not determined that the additional medical questionnaire C2 has been received from the user terminal 10 (No at step S27), step S27 is repeated.

When it is determined that the additional medical questionnaire C2 has been received from the user terminal 10 at step S27, the predicted disease name identifying function 207 re-predicts the disease name(s) of the patient at the subsequent step S28.

Subsequently, the medical interview information generating function 208 generates the medical interview information 35 (step S29).

The medical interview information generating function 208 then makes the communication control function 210 transmit the medical interview information 35 to the doctor terminal 4 (step S30). After that, the server apparatus 2 terminates the process in FIG. 10.

Explanation of the Processing Flow of the Doctor Terminal

The controller 101 of the doctor terminal 4 determines whether the medical interview information 35 is received (step S40). When the controller 101 determines that the medical interview information 35 has been received (Yes at step S40), the process proceeds to step S41. When the controller 101 does not determine that the medical interview information 35 has been received (No at step S40), step S40 is repeated.

When determining that the medical interview information 35 has been received at step S40, the controller 101 of the doctor terminal 4 displays the medical interview information 35 on the display 107 (step S41). The doctor terminal 4 then terminates the process in FIG. 10.

As described above, in the server apparatus 2 (the medical interview apparatus) of the embodiment, the first receiving function 202 receives the answer to the initial medical questionnaire C1 inquiring the symptoms. The disease name predicting function 203 predicts at least one disease name based on the symptom(s) received by the first receiving function 202. The symptom predicting function 204 predicts the symptom(s) of the disease name(s) predicted by the disease name predicting function 203 based on the predicted disease name(s). The medical questionnaire regenerating function 205 (the generating part) generates the additional medical questionnaire C2 inquiring the symptom(s) to be asked again in accordance with the symptom(s) predicted by the symptom predicting function 204. The second receiving function 206 receives the answer to the additional medical questionnaire C2 generated by the medical questionnaire regenerating function 205. The predicted disease name identifying function 207 identifies the predicted disease name(s) predicted by the disease name predicting function 203 based on the symptom(s) received by the second receiving function 206. That is, the medical interview is performed again using the additional medical questionnaire C2 generated in accordance with the answer to the initial medical questionnaire C1 of the patient. Thus, the disease condition of the patient can be efficiently collected. The patient can fill out the initial medical questionnaire C1 and the additional medical questionnaire C2 before arriving at the hospital. Thus, a waiting time at the hospital can be reduced. Moreover, the medical staff do not need to collect the symptom information of the patient at the hospital.

The medical interview information generating function 208 may guide the user terminal 10 which department to visit based on the result of the additional medical questionnaire C2. This is achieved by registering a department associated with an individual disease name in the above disease name symptom database DB. The medical interview information generating function 208 may also guide the user terminal 10 to call an ambulance based on the result of the additional medical questionnaire C2 if the patient has emergent symptoms.

In the server apparatus 2 (the medical interview apparatus) of the embodiment, the disease name predicting function 203 predicts the predicted disease name(s) by using the disease name prediction model M1 storing the association between an individual symptom and a disease name causing the symptom. Thus, the disease name(s) can be easily and efficiently predicted. By updating the disease name prediction model M1, the disease name predicting function 203 can easily respond to a newly registered disease name.

In the server apparatus 2 (the medical interview apparatus) of the embodiment, the symptom predicting function 204 predicts the symptom(s) by using the symptom prediction model M2 storing the association between an individual disease name and a symptom corresponding to the disease name. Thus, the symptom(s) can be easily and efficiently predicted. By updating the symptom prediction model M2, the symptom predicting function 204 can easily respond to a newly registered symptom.

In the server apparatus 2 (the medical interview apparatus) of the embodiment, the symptom prediction model M2 is the information storing the association of an individual disease name, a symptom corresponding to the disease name, and information indicating certainty of association between the disease name and the symptom. When the disease name predicting function 203 predicts a plurality of predicted disease names, the medical questionnaire regenerating function 205 (the generating part) generates the additional medical questionnaire including a question regarding a symptom predicted to have a highest onset possibility out of symptoms predicted from the respective predicted disease names by the symptom predicting function 204 based on the symptom prediction model M2. Thus, the disease name candidates can be narrowed down by an answer to the question in the additional medical questionnaire C2.

In the server apparatus 2 (the medical interview apparatus) of the embodiment, when the disease name predicting function 203 predicts a plurality of predicted disease names, the medical questionnaire regenerating function 205 (the generating part) generates the additional medical questionnaire C2 including a question regarding a symptom more shared among the predicted disease names out of symptoms predicted from the respective predicted disease names by the symptom predicting function 204. Thus, the disease name candidate having a high possibility can be selected by an answer to the question in the additional medical questionnaire C2.

In the server apparatus 2 (the medical interview apparatus) of the embodiment, when the disease name predicting function 203 predicts a plurality of predicted disease names, the medical questionnaire regenerating function 205 (the generating part) generates the additional medical questionnaire C2 including a question regarding a symptom less shared among the predicted disease names out of symptoms predicted from the respective predicted disease names by the symptom predicting function 204. Thus, the disease name candidates can be narrowed down by an answer to the question in the additional medical questionnaire C2.

In the server apparatus 2 (the medical interview apparatus) of the embodiment, the medical questionnaire regenerating function 205 (the generating part) generates the additional medical questionnaire C2 in which the symptom(s) predicted by the symptom predicting function 204 is highlighted. Thus, the patient easily notices the symptom(s) when answering the additional medical questionnaire C2.

In the server apparatus 2 (the medical interview apparatus) of the embodiment, the medical interview information generating function 208 generates the medical interview information 35 including the answers to the initial medical questionnaire C1 and the additional medical questionnaire C2, and the disease name(s) identified by the predicted disease name identifying function 207. The display control function 209 displays the medical interview information 35 on the doctor terminal 4 to which the doctor inputs the medical record information. Thus, the doctor examining the patient can easily check the medical interview result of the patient.

In the server apparatus 2 (the medical interview apparatus) of the embodiment, the medical interview information 35 highlights the symptom(s) characterizing the diseased name(s) identified by the predicted disease name identifying function 207. Thus, the doctor examining the patient can more easily check the medical interview result of the patient. This improves the efficiency of the medical examination.

The medical interview system 1 of the embodiment allows the server apparatus 2 and the user terminal 10 to work together, thereby collecting the symptoms of the patient. To be more specific, in the server apparatus 2, the first receiving function 202 receives the answer to the initial medical questionnaire C1 inquiring the symptoms. The disease name predicting function 203 predicts at least one predicted disease name based on the symptom(s) received by the first receiving function 202. The symptom predicting function 204 predicts the symptom(s) of the disease name(s) predicted by the disease name predicting function 203 based on the predicted disease name(s). The medical questionnaire regenerating function 205 (the generating part) generates the additional medical questionnaire C2 inquiring the symptom(s) to be asked again in accordance with the symptom(s) predicted by the symptom predicting function 204. The second receiving function 206 receives the answer to the additional medical questionnaire C2 generated by the medical questionnaire regenerating function 205. The predicted disease name identifying function 207 identifies the predicted disease name(s) predicted by the disease name predicting function 203 based on the symptom(s) received by the second receiving function 206. At this point, in the user terminal 10, the communication I/F 103 (the receiver) receives the initial medical questionnaire C1 and the additional medical questionnaire C2 from the server apparatus 2. The display 107 displays the initial medical questionnaire C1 and the additional medical questionnaire C2. The controller 101 (the receiver) receives the answers to the initial medical questionnaire C1 and the additional medical questionnaire C2. The communication I/F 103 (the transmitter) transmits the answers to the initial medical questionnaire C1 and the additional medical questionnaire C2 to the server apparatus 2. Thus, the disease condition of the patient can be efficiently collected.

### Second Embodiment

Next, a medical interview apparatus and a medical interview system according to a second embodiment will be described. A typical electronic medical record describes information such as a past medical history of a patient, a history of present illness, and a medicine that the patient regularly takes. The medical interview apparatus and the medical interview system according to the second embodiment generate a more accurate additional medical questionnaire based on the description in the electronic medical record and the disease name(s) predicted from the answer to the initial medical questionnaire.

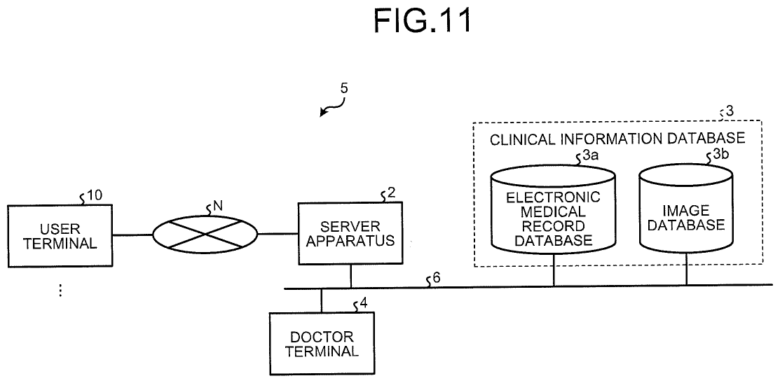

FIG. 11 is a view for explaining a medical interview system 5 according to the second embodiment. The medical interview system 5 differs from the medical interview system 1 of the first embodiment in FIG. 1 in further including a clinical information database 3.

As illustrated in FIG. 11, the clinical information database 3 includes an electronic medical record database 3a, and an image database 3b. Clinical information means the information described in the electronic medical record, information related to the electronic medical record (e.g., nursing records, meal information, and medical payment information), image data, or the like, and is managed for an individual patient.

The electronic medical record database 3a is a database storing and managing an electronic medical record and information related to the electronic medical record for an individual patient. The electronic medical record database 3a of the present embodiment includes a database as a hospital information system (HIS).

The image database 3b is a database storing and managing image data acquired by various modalities and information related to the image data for an individual patient. The image database 3b of the present embodiment includes a database as a picture archiving and communication system (PACS) and a radiology information system (RIS).

The server apparatus 2 acquires the information described in the electronic medical record of the target patient from the electronic medical record database 3a via a network based on the patient information received from the user terminal 10. To be more specific, the symptom predicting function 204 of the server apparatus 2 acquires the information described in the electronic medical record of the target patient from the electronic medical record database 3a via a network based on the patient information received from the user terminal 10.

The information described in the electronic medical record of the patient, acquired by the symptom predicting function 204 of the server apparatus 2 is, for example, the past medical history of the patient, the history of present illness, the medicine that the patient regularly takes, and information regarding medical interviews performed in the past for the patient by doctors (medical interview contents and corresponding answers of the patient), described in the electronic medical record.

The symptom predicting function 204 of the server apparatus 2 predicts at least one symptom based on the disease name(s) predicted by the disease name predicting function 203, and the information described in the electronic medical record of the patient, acquired from the electronic medical record database 3a, by using the symptom prediction model M2.

The symptom prediction model M2 is a computer program achieving an AI model such as a neural network. That is, the symptom prediction model M2 is, for example, a pre-trained model (such as a deep neural network) outputting one or more symptoms for at least one disease name and the information described in the electronic medical record of the patient, input thereto. The computer program achieving the symptom prediction model M2 can be constructed by machine learning using a plurality of sets of training data having a combination of a disease name diagnosed by a doctor, information described in an electronic medical record, and at least one symptom as a typical chief complaint of a patient having the disease name. The symptom predicting function 204 of the server apparatus 2 may predict at least one symptom based on the initial medical questionnaire as well if necessary.

The medical questionnaire regenerating function 205 of the server apparatus 2 generates the additional medical questionnaire C2 based on the at least one symptom predicted by the symptom predicting function 204. The medical questionnaire regenerating function 205 of the server apparatus 2 may generate the additional medical questionnaire C2 in which information overlapped with that of the initial medical questionnaire C1 is distinguished in information included in the additional medical questionnaire C2 by referring to the initial medical questionnaire C1.

The symptom predicting function 204 and the medical questionnaire regenerating function 205 have been described as separate elements in the present embodiment. The symptom predicting function 204 and the medical questionnaire regenerating function 205 may be achieved by one pre-trained model (such as a deep neural network). Such a pre-trained model can be constructed by machine learning using a plurality of sets of training data where a disease name diagnosed by a doctor, information described in an electronic medical record, and the initial medical questionnaire C1 are input data, and at least one symptom not overlapped with those of the initial medical questionnaire C1 is output data (training data).

As described above, in the server apparatus 2 as the medical interview apparatus according to the present embodiment, the symptom predicting function 204 predicts at least one symptom based on the disease name(s) predicted by the disease name predicting function 203 and the information described in the electronic medical record of the patient, acquired from the electronic medical record database 3a. The medical questionnaire regenerating function 205 of the server apparatus 2 generates the additional medical questionnaire C2 based on the at least one symptom predicted by the symptom predicting function 204. Thus, the server apparatus 2 can more accurately generate the additional medical questionnaire C2 based on the information described in the electronic medical record of the patient as well. The server apparatus 2 can also more accurately predict the disease name(s) by using the accurate additional medical questionnaire C2 based on the electronic medical record of the patient.

First Modification

The second embodiment employs the example in which the symptom predicting function 204 of the server apparatus 2 uses the information described in the electronic medical record of the patient, acquired from the electronic medical record database 3a when predicting at least one symptom. The information described in the electronic medical record of the patient, acquired from the electronic medical record database 3a can be also used as necessary, for example, when the disease name predicting function 203 predicts the disease name(s), or when the predicted disease name identifying function 207 identifies the disease name(s).

Second Modification

The above respective embodiments employ the example in which the server apparatus 2 is installed in a hospital. Other than the hospital, the server apparatus 2 may be also installed in any place that allows the server apparatus 2 to communicate with the user terminal 10 and the doctor terminal 4 via a network. For example, the server apparatus 2 can be installed as a server on a cloud.

Third Modification

The above respective embodiments employ the example in which the server apparatus 2 of the medical interview system 1 functions as the medical interview apparatus. The medical interview system 1 may be also achieved, for example, by installing, in the user terminal 10, an application for executing the processing executed in the server apparatus 2.

The term “processor” used in the above description refers to, for example, central processing units (CPUs), graphics processing units (GPUs), or circuits such as application specific integrated circuits (ASICs) and programmable logic devices including simple programmable logic devices (SPLDs), complex programmable logic devices (CPLDs), and field programmable gate arrays (FPGAs). When the processor is, for example, a CPU, the processor reads and executes computer programs stored in a storage circuit to achieve the functions. When the processor is, for example, an ASIC, the functions are directly incorporated into the circuit of the processor as a logic circuit instead of storing computer programs in a storage circuit. The respective processors of the embodiments do not necessarily have to be configured as a single circuit individually. A plurality of independent circuits may be combined to form a single processor so as to achieve the functions. Additionally, the plurality of constituent elements in FIG. 3 may be integrated into one processor to achieve the functions.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical interview apparatus comprising:
processing circuitry configured to
receive an initial answer to an initial medical questionnaire inquiring a symptom;
present an additional medical questionnaire inquiring a symptom related to the initial answer based on the initial answer;
receive an additional answer to the additional medical questionnaire; and
determine at least one first disease name based on the initial answer and the additional answer.

2. The medical interview apparatus according to claim 1, wherein the processing circuitry is configured to
determine at least one second disease name based on the initial answer; and
generate the additional medical questionnaire based on the determined at least one second disease name.

3. The medical interview apparatus according to claim 2, wherein the processing circuitry is configured to determine the at least one first disease name by using a table in which an individual symptom and a disease name causing the symptom correspond to each other.

4. The medical interview apparatus according to claim 2, wherein the processing circuitry is configured to determine the at least one first disease name by using a pre-trained model outputting at least one disease name for at least one symptom input thereto.

5. The medical interview apparatus according to claim 2, wherein the processing circuitry is configured to determine the at least one first disease name by using a pre-trained model outputting at least one disease name for at least one symptom and information described in an electronic medical record of a target patient, input thereto.

6. The medical interview apparatus according to claim 2, wherein the processing circuitry is configured to determine the at least one second disease name by using a table in which an individual symptom and a disease name causing the symptom correspond to each other.

7. The medical interview apparatus according to claim 2, wherein the processing circuitry is configured to determine the at least one second disease name by using a pre-trained model outputting at least one second disease name for at least one symptom input thereto.

8. The medical interview apparatus according to claim 2, wherein the processing circuitry is configured to determine the at least one second disease name by using a pre-trained model outputting at least one second disease name for at least one symptom and information described in an electronic medical record of a target patient, input thereto.

9. The medical interview apparatus according to claim 2, wherein the processing circuitry is configured to generate the additional medical questionnaire by an arithmetic operation using a table in which a plurality of disease names and a plurality of medical interviews correspond to each other.

10. The medical interview apparatus according to claim 2, wherein the processing circuitry is configured to generate the additional medical questionnaire by using a pre-trained model outputting a medical questionnaire for the determined at least one second disease name input thereto.

11. The medical interview apparatus according to claim 2, wherein the processing circuitry is configured to generate the additional medical questionnaire by using a pre-trained model outputting a medical questionnaire for the determined at least one second disease name and information described in an electronic medical record of a target patient, input thereto.

12. The medical interview apparatus according to claim 2, wherein the processing circuitry is configured to generate the additional medical questionnaire including a question regarding a symptom less shared among a plurality of the determined second disease names out of symptoms related to the respective second disease names.

13. The medical interview apparatus according to claim 2, wherein the processing circuitry is configured to generate the additional medical questionnaire including a question regarding a symptom more shared among a plurality of the determined second disease names out of symptoms related to the respective second disease names.

14. The medical interview apparatus according to claim 2, wherein the processing circuitry is configured to generate the additional medical questionnaire in which a predicted symptom is highlighted.

15. The medical interview apparatus according to claim 1, wherein the processing circuitry is configured to:
generate medical interview information including the answers to the initial medical questionnaire and the additional medical questionnaire, and the first disease name; and
transmit the medical interview information to a doctor terminal to which a doctor inputs medical record information.

16. A medical interview apparatus comprising:
processing circuitry configured to
acquire chief complaint information regarding a symptom complained of by a patient, and a disease name corresponding to the chief complaint information of the patient; and
determine information regarding an additional medical interview for the patient by using a pre-trained model outputting information regarding an additional medical interview based on the chief complaint information and the disease name.

17. A medical interview apparatus comprising:
processing circuitry configured to
receive an initial answer to an initial medical questionnaire inquiring a symptom;
determine at least one first disease name based on the initial answer;
present an additional medical questionnaire inquiring a symptom related to the at least one first disease name based on the at least one first disease name;
receive an additional answer to the additional medical questionnaire; and
determine at least one second disease name based on the initial answer and the additional answer.
